(19) United States
(12) Patent Application Publication
(10) Pub. No.: US 2010/0137703 A1
(43) Pub. Date: Jun. 3, 2010

(54) BLOOD FLOW DYNAMIC ANALYSIS APPARATUS AND MAGNETIC RESONANCE IMAGING SYSTEM

(76) Inventor: Hiroyuki Kabasawa, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/625,246

(22) Filed: Nov. 24, 2009

(30) Foreign Application Priority Data
Nov. 28, 2008 (JP) 2008-304066

(51) Int. Cl. A61B 5/055

(52) U.S. Cl. 600/419

(57) ABSTRACT

A blood flow dynamic analysis apparatus for determining a baseline indicative of a signal strength prior to an arrival of a contrast agent to a predetermined region of a subject, based on MR signals collected in time series from the predetermined region of the subject with the contrast agent injected therein, includes a time detection unit for detecting a time of data minimal in signal strength, of a first data sequence in which data of signal strengths of the MR signals are arranged in time series, a data fetch unit for fetching a second data sequence which appears prior to the time detected by the time detection unit, from within the first data sequence, a data detection unit for detecting centrally-located data from within a third data sequence obtained by sorting the second data sequence in the order of magnitudes of the signals strengths, a data extraction unit for extracting data from the third data sequence, based on the centrally-located data, and a baseline determination unit for determining the baseline, based on the data extracted by the data extraction unit.

### CROSS REFERENCE TO RELATED APPLICATIONS

This application claims the benefit of Japanese Patent Application No. 2008-304066 filed Nov. 28, 2008, which is hereby incorporated by reference in its entirety.

### BACKGROUND OF THE INVENTION

The embodiments described herein relate to a blood flow dynamic analysis apparatus for analyzing a blood flow dynamic state, and a magnetic resonance imaging system having the blood flow dynamic analysis apparatus.

As a method for performing a diagnosis of brain infarction, there is known a method using a contrast agent. In order to carry out the diagnosis of the brain infarction using the contrast agent, the contrast agent is injected into a subject and MR signals are collected from slices set to the subject on a time-series basis. Thereafter, there is a need to determine a baseline indicative of a signal strength of each MR signal prior to the arrival of the contrast agent for each of regions lying in each slice. The baseline is a parameter essential for calculation of a change ΔR2* in transverse relaxation velocity or rate of each spin, and the like at the time that the contrast agent has passed through each region of the slice. Although a method for determining the baseline manually and a method for determining it automatically are known, the method for determining the baseline automatically has been in widespread use because it is necessary to carry out the diagnosis of the brain infarction promptly in a short period of time (refer to Japanese Unexamined Patent Publication No. 2004-57812).

The method of described above is however accompanied by the problem that when an S/N ratio of each MR signal is small, the accuracy of a calculated value of the baseline is degraded.

### BRIEF DESCRIPTION OF THE INVENTION

A blood flow dynamic analysis apparatus for determining a baseline indicative of a signal strength prior to an arrival of a contrast agent to a predetermined region of a subject, based on MR signals collected in time series from the predetermined region of the subject with the contrast agent injected therein, includes a time detection unit for detecting a time of data minimal in signal strength, of a first data sequence in which data of signal strengths of the MR signals are arranged in time series; a data fetch unit for fetching a second data sequence which appears prior to the time detected by the time detection unit, from within the first data sequence; a data detection unit for detecting centrally-located data from within a third data sequence obtained by sorting the second data sequence in the order of magnitudes of the signals strengths; a data extraction unit for extracting data from the third data sequence, based on the centrally-located data; and a baseline determination unit for determining the baseline, based on the data extracted by the data extraction unit.

A magnetic resonance imaging system of the invention is equipped with the blood flow dynamic analysis apparatus of the invention.

A second data sequence that appears prior to the time of data minimal in signal strength is fetched from within a first data sequence arranged in time series. The second data sequence is sorted in the order of magnitude of the signal strength. Thereafter, centrally-located data is detected from the data sorted in the order of the magnitude of the signal strength. There is a tendency that when the data are sorted in the order of magnitude of the signal strength, data usable for determination of a baseline concentrate on the neighborhood of the center of the sorted data. Thus, the accuracy of the calculated value of the baseline can be enhanced even though the SN ratio of each MR signal is small, by using the data located in the center.

Further objects and advantages of the present invention will be apparent from the following description of the preferred embodiments of the invention as illustrated in the accompanying drawings.

### BRIEF DESCRIPTION OF THE DRAWINGS

FIG. 1 is a schematic diagram of a magnetic resonance imaging system 1 according to one embodiment of the invention.

FIG. 2 is a diagram showing a processing flow of the magnetic resonance imaging system.

FIG. 3 is one example illustrative of slices set to a subject 8.

FIGS. 4A, 4B, and 4C are conceptual diagrams showing frame images obtained from their corresponding slices S1 through Sn.

FIGS. 5A and 5B are diagrams showing changes in signal strength with respect to time in a sectional area of a slice Sk set to a head 8a of the subject 8.

FIG. 6 is a diagram showing a data sequence DS2 fetched from within a data sequence DS1.

FIG. 7 is a diagram showing sorted data D1 through D24.

FIG. 8 is a diagram showing the positions of a lower limit value LC1 and an upper limit value UC1.

FIG. 9 is a diagram showing a confidence interval CI.

FIG. 10 is a diagram for showing labeled data of a data sequence DS2 arranged in time series.

FIG. 11 is a diagram showing a baseline BL and an arrival time AT.

FIGS. 12A and 12B are diagrams showing one example of another method for determining an arrival time AT.

### DETAILED DESCRIPTION OF THE INVENTION

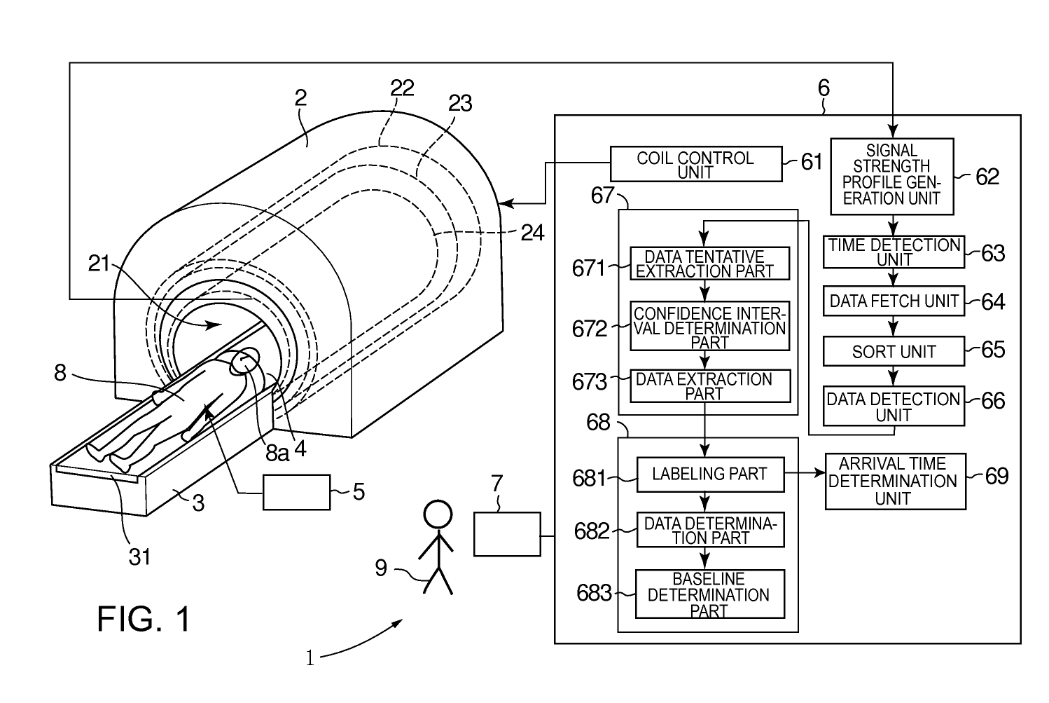

FIG. 1 is a schematic diagram of a magnetic resonance imaging system 1 according to one embodiment of the invention.

The magnetic resonance imaging system (hereinafter called MRI (Magnetic Resonance Imaging) system) 1 has a coil assembly 2, a table 3, a reception coil 4, a contrast agent injection device 5, a control device 6 and an input device 7.

The coil assembly 2 has a bore 21 that accommodates a subject 8 therein, a superconducting coil 22, a gradient coil 23 and a transmission coil 24. The superconducting coil 22 applies a static magnetic field B0, the gradient coil 23 applies a gradient pulse and the transmission coil 24 transmits an RF pulse.

The table 3 has a cradle 31. The cradle 31 is configured so as to move in a z direction and a −z direction. With the movement of the cradle 31 in the z direction, the subject 8 is carried in the bore 21. With the movement of the cradle 31 in the −z direction, the subject 8 carried in the bore 21 is carried out from the bore 21.

The contrast agent injection device 5 injects a contrast agent into the subject 8.

The reception coil 4 is attached to the head 8a of the subject 8. An MR (Magnetic Resonance) signal received by the reception coil 4 is transmitted to the control device 6.

The control device 6 has coil control unit 61 through arrival time determination unit 69.

The coil control unit 61 controls the transmission coil 24 and the gradient coil 23 in such a manner that a pulse sequence for imaging the subject 8 is executed in response to an imaging command of the subject 8, which has been inputted from the input device 7 by an operator 9.

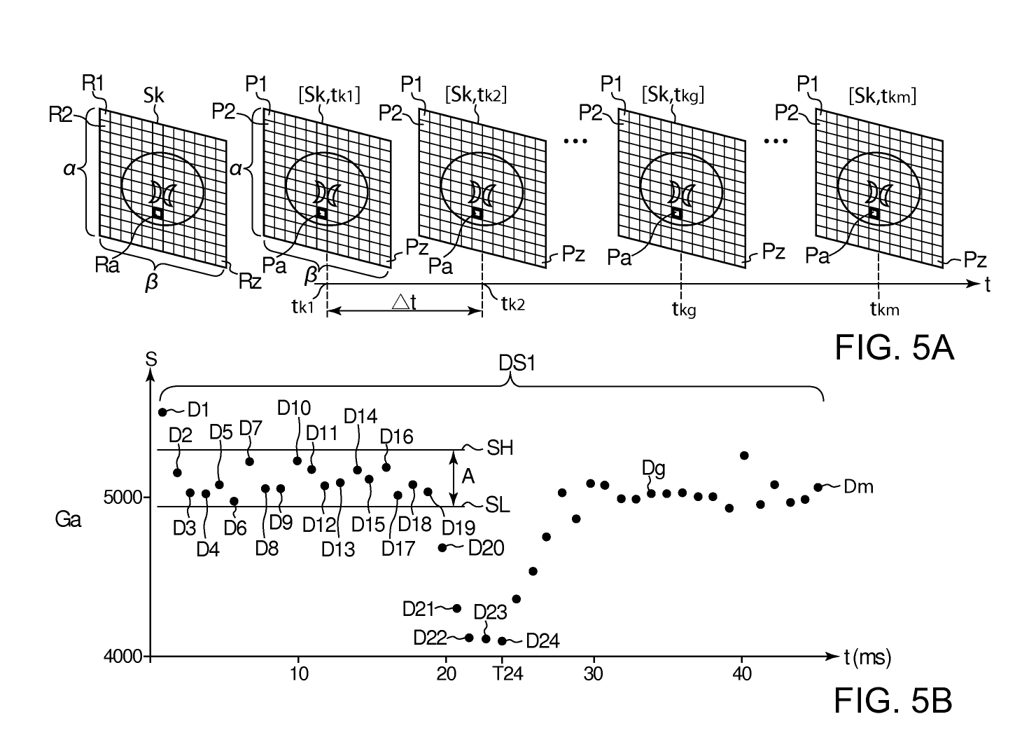

The signal strength profile generation unit 62 generates a signal strength profile Ga of a data sequence DS1 (refer to FIGS. 5A and 5B).

The time detection unit 63 detects a time T24 at data D24 minimal in signal strength S, of the data sequence DS1 (refer to FIG. 5B).

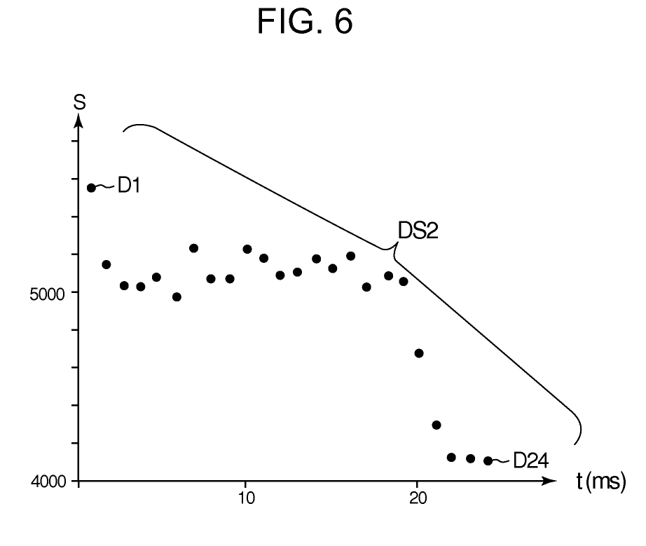

The data fetch unit 64 fetches a data sequence DS2 (refer to FIG. 6) from within the data sequence DS1 (refer to FIG. 5B) arranged in time series.

The sort unit 65 rearranges or sorts the data sequence DS2 in the order of magnitude of each signal strength.

The data detection unit 66 detects data D24 minimal in signal strength from within a data sequence DS3 arranged in the order of magnitude of the signal strength. Further, the data detection unit 66 also detects data located in the center of the data sequence DS3 arranged in the order of magnitude of the signal strength from within the data sequence DS3.

The data extraction unit 67 has a data tentative extraction part 671, a confidence interval determination part 672 and a data extraction part 673.

The data tentative extraction part 671 tentatively extracts data from within the data sequence DS3 arranged in the order of magnitude of the signal strength, based on the data detected by the data detection unit 66.

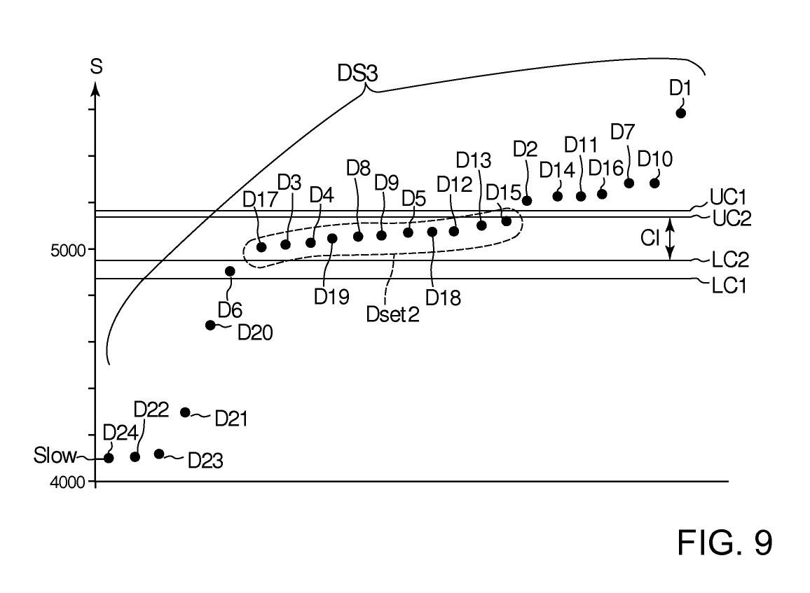

The confidence interval determination part 672 determines a confidence interval CI at which data fitted to determine a baseline BL exist with respect to a set Dset1 of the data tentatively extracted by the data tentative extraction part 671 (refer to FIG. 9).

The data extraction part 673 extracts a set Dset2 of data contained in the confidence interval CI from within the set Dset1 of the tentatively extracted data (refer to FIG. 9).

The baseline determination unit 68 has a labeling part 681, a data determination part 682 and a baseline determination part 683.

The labeling part 681 labels data corresponding to the data (refer FIG. 9) extracted from the confidence interval CI of the data sequence DS3, of the data (refer to FIG. 6) contained in the data sequence DS2 arranged in time series.

The data determination part 682 determines data used to determine the baseline BL, based on the data labeled by the labeling part 681.

The baseline determination part 683 determines the baseline BL, based on the data determined by the data determination part 682.

The arrival time determination unit 69 determines an arrival time AT, based on the data labeled by the labeling part 681.

The input device 7 inputs various commands to the control device 6 in accordance with the operation of the operator 9.

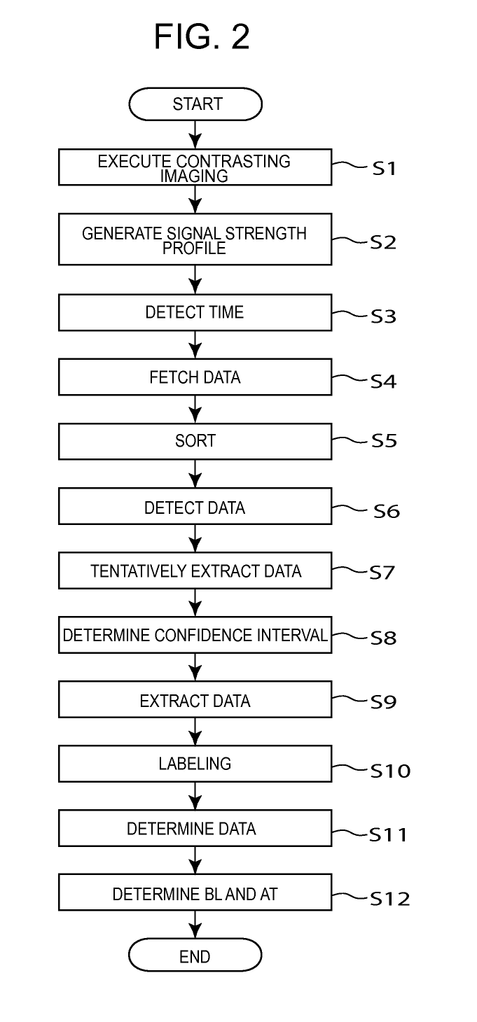

FIG. 2 is a diagram showing a processing flow of the magnetic resonance imaging system 1.

At Step S1, contrast-enhanced or contrasting imaging is performed on the head 8a of the subject 8. The operator manipulates the input device 7 to set slices to the subject 8.

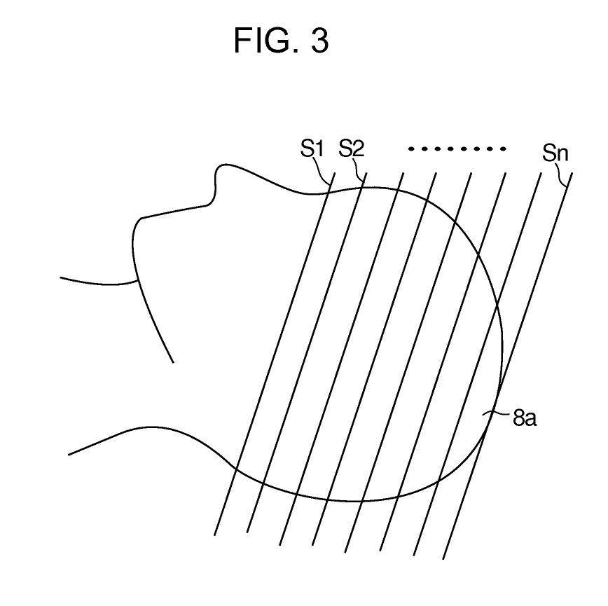

FIG. 3 is one example illustrative of slices set to the subject 8.

n sheets of slices S1 through Sn are set to the subject 8. The number of slices is, for example, n=12. The number of the slices can be set to an arbitrary number of sheets as needed. An imaging area of the head 8a of the subject 8 is determined for each of the slices S1 through Sn.

After the slices S1 through Sn have been set, the operator 9 transmits a contrast agent injection command to the contrast agent injection device 5 and transmits a command for imaging or obtaining the subject 8 to the coil control unit 61 of the MRI system (refer to FIG. 1). The coil control unit 61 controls the transmission coil 24 and the gradient coil 23 in such a manner that a pulse sequence for imaging the head 8a of the subject 8 in response to the corresponding imaging command.

In the present embodiment, a pulse sequence for obtaining m sheets of continuously-captured frame images from their corresponding slices is executed by a multi-slice scan. Thus, the m sheets of frame images are obtained per slice. For example, the number of frame images m=85. With the execution of the pulse sequence, data are collected from the head 8a of the subject 8.

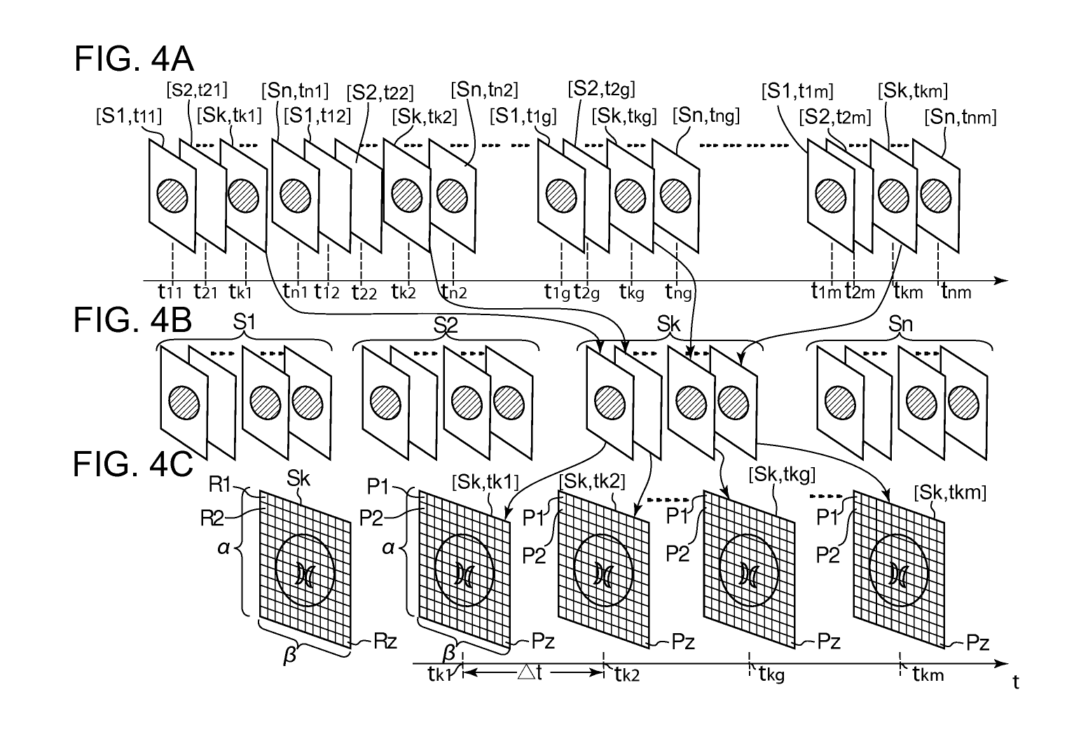

FIGS. 4A, 4B, and 4C are conceptual diagrams showing frame images obtained from their corresponding slices S1 through Sn.

FIG. 4A is a schematic diagram showing that the n sheets of slices S1 through Sn set to the head 8a of the subject 8 are arranged in time series in accordance with the order of collection thereof, FIG. 4B is a schematic diagram showing the manner in which the frame images of FIG. 4A are classified for each of the slices S1 through Sn, and FIG. 4C is a schematic diagram showing frame images collected or acquired from the slice Sk, respectively.

Frame images [S1, t11] through [Sn, tnm] are acquired from the slices S1 through Sn (refer to FIG. 3) set to the head 8a of the subject 8 (refer to FIG. 4A). In FIG. 4A, the left character of [,] indicative of each frame image represents a slice at which each frame image is acquired, and the right character thereof represents the time at which each frame image is acquired.

FIG. 4B shows the manner in which the frames images shown in FIG. 4A are classified for each of the slices S1 through Sn. FIG. 4B shows by arrows, to which frame images of the frame images [S1, t11] through [Sn, tnm] arranged in time series in FIG. 4A the frame images [Sk, tk1] through [Sk, tkm] of the slice Sk of the slices S1 through Sn correspond respectively.

The section of the slice Sk and the m sheets of frame images [Sk, tk1] through [Sk, tkm] acquired from the slice Sk are shown in FIG. 4C. The section of the slice Sk is divided into α×β regions R1, R2, . . . Rz. The frame images [Sk, tk1] through [Sk, tkm] have α×β pixels P1, P2, . . . Pz respectively. The pixels P1, P2, . . . Pz of the frame images [Sk, tk1] through [Sk, tkm] are equivalent to those obtained by imaging or obtaining the regions R1, R2, . . . Rz of the slice Sk at times tk1 through tkm (time intervals Δt).

Incidentally, while only the frame images obtained at the slice Sk are shown in FIG. 4C, m sheets of frame images are acquired even at other slices in a manner similar to the slice Sk.

After the execution of Step S1, the processing flow proceeds to Step S2.

At Step S2, the signal strength profile generation unit 62 (refer to FIG. 1) generates a profile of a data sequence DS1 (refer to FIGS. 5A and 5B). A description will hereinafter be made of how the signal strength profile generation unit 62 generates the profile of the data sequence DS1, with reference to FIGS. 5A and 5B.

FIGS. 5A and 5B are diagrams showing changes in signal strength with time in a sectional area of the slice Sk set to the head 8a of the subject 8.

The section of the slice Sk of the subject 8 and the frame images [Sk, tk1] through [Sk, tkm] of the slice Sk are shown in FIG. 5A (refer to FIG. 4C).

A schematic diagram of a signal strength profile Ga indicative of changes in signal strength with time at a region Ra of the slice Sk is shown in FIG. 5B.

The horizontal axis indicates the time t at which each of the frame images [Sk, tk1] through [Sk, tkm] is acquired from the slice Sk. The vertical axis indicates the signal strength S at each of pixels Pa of the frame images [Sk, tk1] through [Sk, tkm]. Each of the pixels Pa of the frame images [Sk, tk1] through [Sk, tkm] is equivalent to one obtained by capturing or imaging the region Ra of the slice Sk at each of the times tk1 through tkm. The signal strength profile Ga shows a data sequence DS1 in which data D1 through Dm are arranged on a time-series basis. The data D1 through Dm respectively indicate the signal strengths S at the pixels Pa of the frame images [Sk, tk1] through [Sk, tkm]. For example, the data D1 indicates the signal strength S at the pixel Pa of the frame image [Sk, tk1], and the data Dg indicates the signal strength S at the pixel Pa of the frame image [Sk, tkg].

While the signal strength profile Ga at the region Ra of the slice Sk has been shown in FIG. 5B, signal strength profiles Ga are generated or formed even at other regions in the slice Sk. Further, signal strength profiles Ga are generated similarly even at respective regions related to other slices other than the slice Sk.

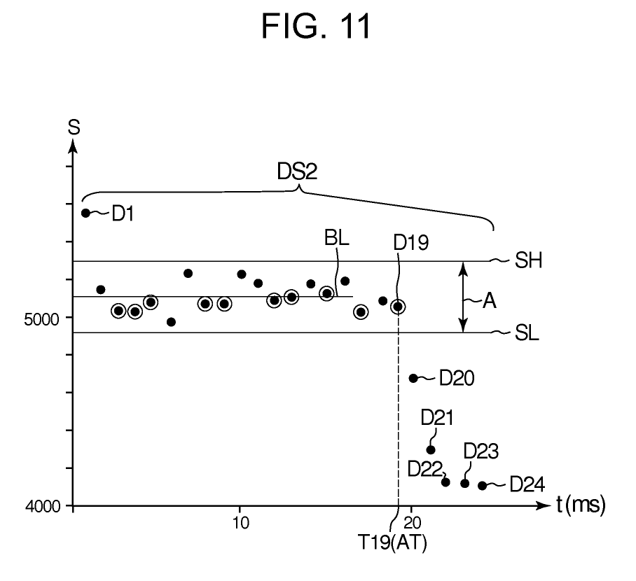

In the present embodiment, a baseline BL (refer to FIG. 11) to be described later is determined from the data sequence DS1 of the signal strength profile Ga. The baseline BL is of a line indicative of a signal strength S prior to the arrival of a contrast agent to the corresponding region Ra of the slice Sk. The baseline BL is a parameter necessary to calculate a change ΔR2* in transverse relaxation velocity or rate of each spin, and the like at the time that the contrast agent has passed through the region Ra of the slice Sk. The baseline BL is set to any position of a range A in which the signal strength S increases and decreases repeatedly in the first half of the signal strength profile Ga. Since, however, the optimal position of the baseline BL varies every signal strength profile Ga, it is necessary to determine the optimal position of the baseline BL every signal strength profile Ga. Thus, in the present embodiment, Steps S3 through S11 are executed in such a manner that the baseline BL can be set to the optimal position. Steps S3 through S11 will be explained below.

At Step S3, the time detection unit 63 (refer to FIG. 1) detects a time T24 at data D24 minimal in signal strength S, of the data sequence DS1 of the signal strength profile Ga (refer to FIG. 5B). After the time T24 has been detected, the processing flow proceeds to Step S4.

At Step S4, the data fetch unit 64 (refer to FIG. 1) fetches such a data sequence DS2 (including the data D24 at the time T24 detected by the time detection unit 63 and data D1 through D23 prior to the time T24) as shown in FIG. 6 from within the data sequence DS1 arranged in time series.

FIG. 6 is a diagram showing the data sequence DS2 fetched from within the data sequence DS1.

The data sequence DS2 contains the data D1 through D24. In FIG. 6, only the data D1 and D24 are designated by reference symbols. Reference symbols for other data D2 through D23 are omitted. After the data D1 through D24 have been fetched, the processing flow proceeds to Step S5.

At Step S5, the sort unit 65 (refer to FIG. 1) sorts the fetched data sequence DS2 (data D1 through D24) in the order of magnitude of the signal strength.

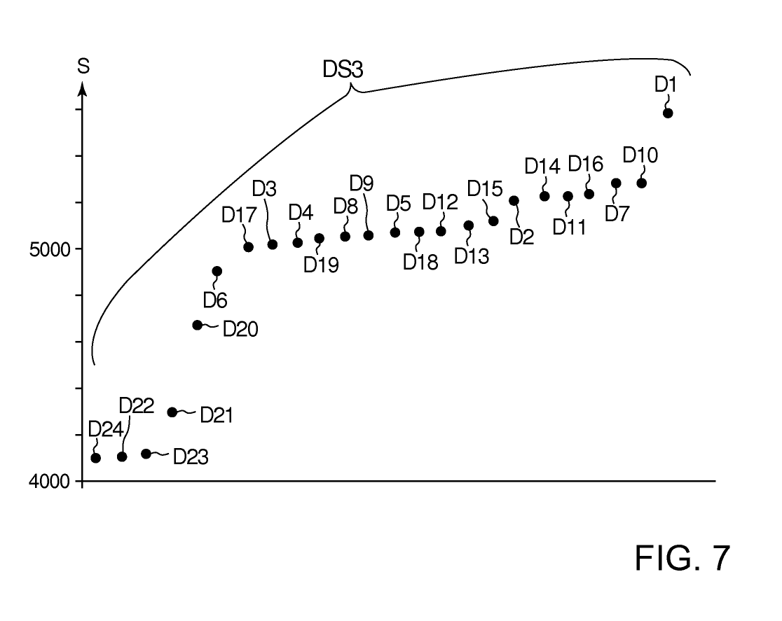

FIG. 7 is a diagram showing the sorted data D1 through D24.

The horizontal axis of a graph indicates the positions of the sorted data D1 through D24, and the vertical axis thereof indicates the signal strength S. With the sorting of the data sequence DS2 (data D1 through D24) in the order of magnitude of the signal strength, a data sequence DS3 arranged in the order of magnitude of the signal strength is obtained. After the data D1 through D24 have been sorted in the order of magnitude of the signal strength S, the processing flow proceeds to Step S6.

At Step S6, the data detection unit 66 (refer to FIG. 1) detects the data D24 minimal in signal strength S from within the data sequence DS3 arranged in the order of magnitude of the signal strength.

Further, the data detection unit 66 detects data located in the center of the data sequence DS3 arranged in the order of magnitude of the signal strength from within the data sequence DS3. In the present embodiment, however, the number of data contained in the data sequence DS3 is 24, i.e., an even number. Thus, the position of the center of the data sequence DS3 becomes a position E between twelfth data D9 as counted from the side small in signal strength S and twelfth data D5 as counted from the side large in signal strength S. However, no data exists in the position E. Therefore, in the present embodiment, the data D9 adjacent to the side small in signal strength S is detected as the data located in the center with respect to the position E. However, the data D5 adjacent to the side large in signal strength S may be detected as the data located in the center. Incidentally, when the number of data is an odd number, data located in the middle thereof is detected as the data located in the center.

The data detection unit 66 detects the data D24 and D9 in the above-described manner. After the data D24 and D9 have been detected, the processing flow proceeds to Step S7.

At Step S7, the data tentative extraction part 671 (refer to FIG. 1) tentatively extracts data likely to be usable for determining a baseline BL from within the data sequence DS3 arranged in the order of magnitude of the signal strength, based on the detected data D24 and D9.

In order to tentatively extract data, the data tentative extraction part 671 first determines a lower limit value LC1 and an upper limit value UC1 of a signal strength S defined as the reference for tentatively extracting the data. The lower limit value LC1 and the upper limit value UC1 are calculated from the following equations:

LC1=Sm1−(Sm1−Slow)×k1 Eq. (1)

UC1=Sm1+(Sm1−Slow)×k2 Eq. (2)

where Sm1 is a signal strength of data D9 located in the center, Slow is a signal strength of data D24, and k1 and k2 are constants.

Thus, the lower limit value LC1 and the upper limit value UC1 are calculated from the equations (1) and (2).

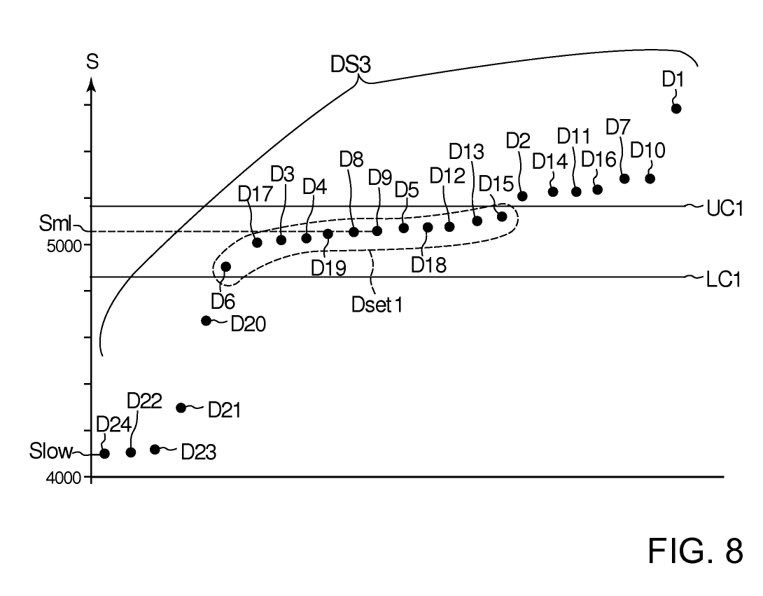

FIG. 8 is a diagram showing the positions of the lower limit value LC1 and the upper limit value UC1.

After the lower limit value LC1 and the upper limit value UC1 have been calculated, a set Dset1 of data (data D6, D17, D3, D4, D19, D9, D5, D18, D12, D13 and D15) located between the lower limit value LC1 and the upper limit value UC1 is tentatively extracted.

Incidentally, the lower limit value LC1 and the upper limit value UC1 depend on the constants k1 and k2 along with 5 ml and Slow (refer to the equations (1) and (2)). The smaller the constants k1 and k2, the narrower the interval between the lower limit value LC1 and the upper limit value UC1. On the other hand, the larger the constants k1 and k2, the wider the interval between the lower limit value LC1 and the upper limit value UC1. Since the number of tentatively extracted data becomes small when the interval between the lower limit value LC1 and the upper limit value UC1 becomes too narrow, there is a need to wide the interval between the lower limit value LC1 and the upper limit value UC1 to some extent in such a manner that a certain number of data can be tentatively extracted. Since, however, the number of the tentatively extracted data increases when the interval between the lower limit value LC1 and the upper limit value UC1 becomes excessively wide, the ratio of the number of data unfitted to determine the baseline BL to the number of the tentatively extracted data also increases. It is thus necessary to set the constants k1 and k2 in such a way that the interval between the lower limit value LC1 and the upper limit value UC1 becomes a proper value. In the present embodiment, the constants are set to k1=k2=0.1. However, the values of k1 and k2 may be set to values other than 0.1 according to imaging conditions.

In the present embodiment, a set Dset1 of data is tentatively extracted. All data contained in the set Dset1 of the tentatively extracted data are also usable as data for determining the baseline BL. There is however a possibility that data undesirable to be used as the data for determining the baseline BL will be contained in the set Dset1 of the data depending on deviations in signal strength between the data contained in the set Dset1 of the tentatively extracted data. Thus, in the present embodiment, the corresponding data used to determine the baseline BL is extracted from within the set Dset1 of the tentatively extracted data. Therefore, the processing flow proceeds to Step S8.

At Step S8, the confidence interval determination part 672 (refer to FIG. 1) determines a confidence interval CI at which the corresponding data fitted to determine the baseline BL is likely to exist with respect to the set Dset1 of the tentatively extracted data. The confidence interval CI is determined according to a lower limit value LC2 and an upper limit value UC2 of a signal strength S. The lower limit value LC2 and the upper limit value UC2 are calculated from, for example, the following equations:

LC2=Sm2−STD×k3 Eq. (3)

UC2=Sm2−STD×k4 Eq. (4)

where Sm2 is an average value of signal strengths of all data contained in set Dset1 of tentatively extracted data, STD is a standard deviation, and k3 and k4 are constants.

Thus, the lower limit value LC2 and the upper limit value UC2 are calculated from the equations (3) and (4).

FIG. 9 is a diagram showing the confidence interval CI.

The lower limit value LC2 and the upper limit value UC2 of the confidence interval CI are located between the lower limit value LC1 and the upper limit value UC1 used when the data is tentatively extracted. As a result, it is understood that data D6 is omitted from the confidence section CI and low in reliability as the data used to determine the baseline BL. A set Dset2 of data (data D17, D3, D4, D19, D8, D9, D5, D18, D12, D13 and D15) is contained in the confidence interval CI.

Incidentally, the lower limit value LC2 and the upper limit value UC2 depend on the constants k3 and k4 along with Sm2 and STD (refer to the equations (3) and (4)). While the values of the constants k3 and k4 take various values according to imaging conditions or the like, the constants are set to k3=k4=3 in the present embodiment. However, the values of the constants k3 and k4 may be set to values other than 3 according to the imaging conditions or the like.

After the confidence interval CI has been determined, the processing flow proceeds to Step S9.

At Step S9, the data extraction part 673 (refer to FIG. 1) extracts the set Dset2 of the data (data D17, D3, D4, D19, D8, D9, D5, D18, D12, D13 and D15) contained in the confidence interval CI from within the set Dset1 of the tentatively extracted data. After the extraction of the data set Dset2, the processing flow proceeds to Step S10.

At Step S10, the labeling part 681 (refer to FIG. 1) labels data corresponding to the data extracted from the confidence interval CI of the data sequence DS3, of the data (refer to FIG. 6) contained in the data sequence DS2 arranged on a time series basis.

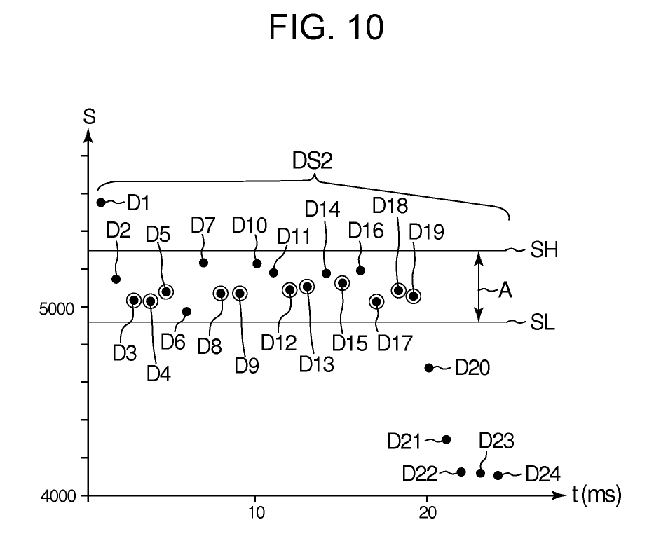

FIG. 10 is a diagram for showing labeled data of the data sequence DS2 arranged in time series. In FIG. 10, the labeled data (D3, D4, D5, D8, D9, D12, D13, D15, D17, D18 and D19) are shown with being surrounded by white circles. It is understood that when FIGS. 10 and 9 are compared, the data contained in the set Dset2 of the data shown in FIG. 9 are labeled in FIG. 10.

It is understood that referring to FIG. 10, the labeled data (D3, D4, D5, D8, D9, D12, D13, D15, D17, D18 and D19) appear in a range A in which an increase/decrease in signal strength is repeated. It is thus understood that the labeled data are data fitted to determine the baseline BL. After the data have been labeled, the processing flow proceeds to Step S9.

At Step S11, the data determination part 682 (refer to FIG. 1) determines data used to determine the baseline BL, based on the labeled data. Referring to FIG. 10, unlabeled data (D2, D6, D7, D10, D11, D14 and D16) also exist in the range A in which the increase/decrease in signal strength is repeated, in addition to the labeled data. However, the unlabeled data (D6, D7, D10, D11, D14 and D16) other than the data D2 are interposed between the labeled data. In such a case, even the unlabeled data ((D6, D7, D10, D11, D14 and D16) are considered to be data fitted to determine the baseline BL. Therefore, the data determination part 682 determines both the labeled data (D3, D4, D5, D8, D9, D12, D13, D15, D17, D18 and D19) and the unlabeled data (D6, D7, D10, D11, D14 and D16) as the data used to determine the baseline BL. Thus, the data determination part 682 determines the data D3 through D19 as the data used to determine the baseline BL. Thereafter, the processing flow proceeds to Step S12.

At Step S12, the baseline determination part 683 (refer to FIG. 1) calculates an average value of signal strengths S of the data D3 through D19 determined by the data determination part 682 and determines the calculated average value as a baseline BL. The arrival time determination unit 69 (refer to FIG. 1) determines a time AT (arrival time) at which the contrast agent has reached the region Ra of the slice Sk, based on the labeled data (D3, D4, D5, D8, D9, D12, D13, D15, D17, D18 and D19)).

FIG. 11 is a diagram sowing a baseline BL and an arrival time AT.

In FIG. 11, reference symbols are omitted for data lying within a range A except for data D19.

It is understood that referring to FIG. 11, the baseline BL is set within the range A in which an increase/decrease in signal strength S is repeated. A time T19 of the data D19 that appears finally on a time-series basis, of labeled data (D3, D4, D5, D8, D9, D12, D13, D15, D17, D18 and D19) is determined as the arrival time AT. It is understood that the signal strength S decreases suddenly from immediately after the data D19, and the time of the data D19 is proper as the arrival time AT.

The procedure for determining the baseline BL and the arrival time AT at the region Ra (refer to FIG. 5A) of the slice Sk has been explained up to now. However, baselines BL and arrival times AT at regions of other slices other than the slice Sk are also determined by an approach similar to above.

In the present embodiment, the data sequence DS2 (refer to FIG. 6) including the data D24 minimal in signal strength and the data D1 through D23 that appear prior to the data D24 is fetched from within the data sequence DS1 (refer to FIG. 5B) arranged in time series. The data sequence DS2 is sorted in the order of magnitude of the signal strength. Thereafter, the data D9 located in the center is detected from within the data D1 through D24 sorted in the order of magnitude of the signal strength. There is a tendency that when they are sorted in the order of magnitude of the signal strength, the data usable for determination of the baseline BL concentrate on the neighborhood of the center of the sorted data (refer to FIG. 9). Thus, the accuracy of the calculated value of the baseline BL can be enhanced even though the SN ratio of an MR signal is large, by determining the data D3 through D19 used to determine the baseline BL finally, based on the data D9 located in the center.

Incidentally, in the present embodiment, the set Dset2 of the data contained in the confidence interval CI is extracted from the set Dset1 of the tentatively extracted data. The data D3 through D19 used to determine the baseline BL are determined based on the data set Dset2. However, the data used to determine the baseline BL may be determined based on the set Dset1 of the tentatively extracted data.

In the present embodiment, the data D1 through D24 are fetched as the data sequence DS2. However, the data D1 through D23 of the data D1 through D24 may be fetched out as the data sequence DS2 without fetching the data 24 minimal in signal strength S.

Although the time T19 of the data D19 is determined as the arrival time AT in the present embodiment, the arrival time AT can also be determined by another method. A description will hereinafter be made of a method for determining the arrival time AT by means of another method.

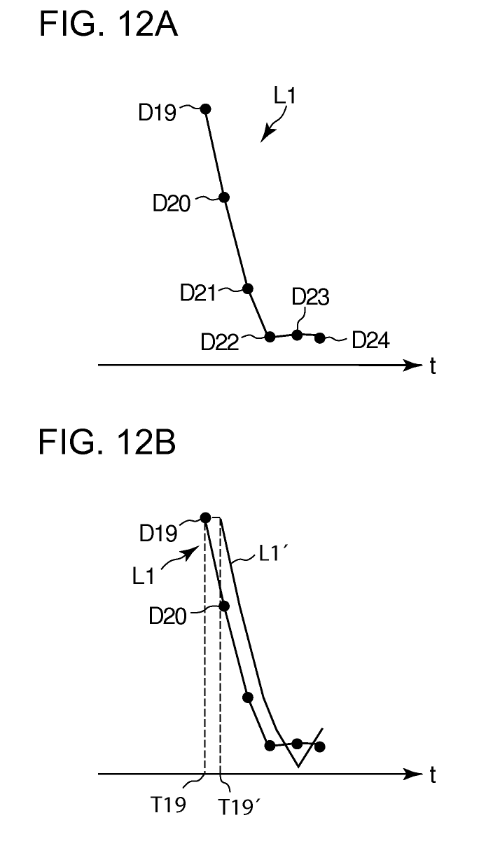

FIGS. 12A and 12B are diagrams showing one example of another method for determining the arrival time AT.

As shown in FIG. 12A, data D19 through D24 are first connected by straight lines and a line L1 for connecting the data D19 through D24 is defined.

Next, as shown in FIG. 12B, the line L1 is fitted using a predetermined function (gamma function or polynomial expression). With this fitting, the line L1 changes to a line L1′. A time T19′ of a position corresponding to the data D19 is calculated from the line L1′. The time T19′ calculated in this way may be determined as the arrival time AT.

Many widely different embodiments of the invention may be configured without departing from the spirit and the scope of the present invention. It should be understood that the present invention is not limited to the specific embodiments described in the specification, except as defined in the appended claims.

1. A blood flow dynamic analysis apparatus configured to determine a baseline indicative of a signal strength prior to an arrival of a contrast agent to a predetermined region of a subject, based on MR signals collected in time series from the predetermined region of the subject said blood flow dynamic analysis apparatus comprising:
a time detection unit configured to detect a time of data having a minimal signal strength, of a first data sequence in which data of signal strengths of the MR signals are arranged in time series;
a data fetch unit configured to fetch a second data sequence which appears prior to the time detected by said time detection unit, from within the first data sequence;
a data detection unit configured to detect centrally-located data from within a third data sequence obtained by sorting the second data sequence in the order of magnitudes of the signals strengths;
a data extraction unit configured to extract data from the third data sequence, based on the centrally-located data; and
a baseline determination unit configured to determine the baseline, based on the data extracted by said data extraction unit.

2. The blood flow dynamic analysis apparatus according to claim 1, wherein said baseline determination unit comprises:
a labeling part configured to label data within the second data sequence that corresponds to the data extracted from the third data sequence;
a data determination part configured to determine data used to determine the baseline, based on the labeled data; and
a baseline determination part configured to determine the baseline, based on the data determined by said data determination part.

3. The blood flow dynamic analysis apparatus according to claim 2, wherein when unlabeled third data exists between labeled first data and labeled second data, said data determination part is configured to determine the third data as the data used to determine the baseline along with the first data and the second data.

4. The blood flow dynamic analysis apparatus according to claim 2, further comprising an arrival time determination unit configured to determine an arrival time at which the contrast agent reaches the predetermined region, based on the labeled data.

5. The blood flow dynamic analysis apparatus according to claim 3, further comprising an arrival time determination unit configured to determine an arrival time at which the contrast agent reaches the predetermined region, based on the labeled data.

6. The blood flow dynamic analysis apparatus according to claim 4, wherein said arrival time determination unit is configured to determine the arrival time using a function for performing a fitting process.

7. The blood flow dynamic analysis apparatus according to claim 5, wherein said arrival time determination unit is configured to determine the arrival time using a function for performing a fitting process.

8. The blood flow dynamic analysis apparatus according to claim 1, wherein said data extraction unit comprises:
a data tentative extraction part configured to tentatively extract data from within the third data sequence, based on the centrally-located data;
a confidence interval determination part configured to determine a confidence interval for the tentatively extracted data; and
a data extraction part configured to extract data contained in the confidence interval from within the tentatively extracted data.

9. The blood flow dynamic analysis apparatus according to claim 8, wherein said confidence interval determination part is configured to calculate an average value of the data extracted from the data extraction part and a standard deviation thereof, and to calculate the confidence interval, based on the average value and the standard deviation.

10. The blood flow dynamic analysis apparatus according to claim 1, further comprising a sort unit configured to sort the second data sequence in the order of magnitudes of the signal strengths.

11. The blood flow dynamic analysis apparatus according to claim 8, further comprising a sort unit configured to sort the second data sequence in the order of magnitudes of the signal strengths.

12. The blood flow dynamic analysis apparatus according to claim 1, wherein said data fetch unit is configured to fetch the data at the time detected by said time detection unit from within the first data sequence as the data contained in the second data sequence.

13. The blood flow dynamic analysis apparatus according to claim 8, wherein said data fetch unit is configured to fetch the data at the time detected by said time detection unit from within the first data sequence as the data contained in the second data sequence.

14. A magnetic resonance imaging system comprising;
a contrast injection device configured to inject a contrast into a predetermined region of a subject; and
a blood flow dynamic analysis apparatus configured to determine a baseline indicative of a signal strength prior to an arrival of the contrast agent into the predetermined region, based on MR signals collected in time series from the predetermined region, said blood flow dynamic analysis apparatus comprising:
a time detection unit configured to detect a time of data having a minimal signal strength, of a first data sequence in which data of signal strengths of the MR signals are arranged in time series;
a data fetch unit configured to fetch a second data sequence which appears prior to the time detected by said time detection unit, from within the first data sequence;
a data detection unit configured to detect centrally-located data from within a third data sequence obtained by sorting the second data sequence in the order of magnitudes of the signals strengths;
a data extraction unit configured to extract data from the third data sequence, based on the centrally-located data; and
a baseline determination unit configured to determine the baseline, based on the data extracted by said data extraction part.

15. A magnetic resonance imaging system comprising:
a contrast injection device configured to inject a contrast into a predetermined region of a subject; and
a blood flow dynamic analysis apparatus configured to determine a baseline indicative of a signal strength prior to an arrival of the contrast agent into the predetermined region, based on MR signals collected in time series from the predetermined region, said blood flow dynamic analysis apparatus comprising:
a time detection unit configured to detect a time of data having a minimal signal strength, of a first data sequence in which data of signal strengths of the MR signals are arranged in time series;
a data fetch unit configured to fetch a second data sequence which appears prior to the time detected by said time detection unit, from within the first data sequence;
a data detection unit configured to detect centrally-located data from within a third data sequence obtained by sorting the second data sequence in the order of magnitudes of the signals strengths;
a data tentative extraction part configured to tentatively extract data from within the third data sequence, based on the centrally-located data;
a confidence interval determination part configured to determine a confidence interval for the tentatively extracted data;
a data extraction part configured to extract data contained in the confidence interval from within the tentatively extracted data; and
a baseline determination unit configured to determine the baseline, based on the data extracted by said data extraction unit.

16. The magnetic resonance imaging system according to claim 14, wherein said baseline determination unit comprises:
a labeling part configured to label data within the second data sequence that corresponds to the data extracted from the third data sequence;
a data determination part configured to determine data used to determine the baseline, based on the labeled data; and
a baseline determination part configured to determine the baseline, based on the data determined by said data determination part.

17. The magnetic resonance imaging system according to claim 16, wherein when unlabeled third data exists between labeled first data and labeled second data, said data determination part is configured to determine the third data as the data used to determine the baseline along with the first data and the second data.

18. The magnetic resonance imaging system according to claim 16, further comprising an arrival time determination unit configured to determine an arrival time at which the contrast agent reaches the predetermined region, based on the labeled data.

19. The magnetic resonance imaging system according to claim 15, wherein said baseline determination unit comprises:
a labeling part configured to label data within the second data sequence that corresponds to the data extracted from the third data sequence;
a data determination part configured to determine data used to determine the baseline, based on the labeled data; and
a baseline determination part configured to determine the baseline, based on the data determined by said data determination part.

20. The magnetic resonance imaging system according to claim 19, further comprising an arrival time determination unit configured to determine an arrival time at which the contrast agent reaches the predetermined region, based on the labeled data.
